(19) United States
(12) Patent Application Publication
(10) Pub. No.: US 2012/0289481 A1
(43) Pub. Date: Nov. 15, 2012

(54) COMPOSITIONS AND METHODS FOR TREATING CANCER

(76) Inventors: Jennifer O'NEIL, Dedham, MA (US); Yair BENITA, Brookline, MA (US); Shane MARINE, Perkiomenville, PA (US); Brian HAINES, Boston, MA (US)

(21) Appl. No.: 13/463,951

(22) Filed: May 4, 2012

Related U.S. Application Data
(60) Provisional application No. 61/485,707, filed on May 13, 2011.

(51) Int. Cl. A61K 31/4375; A61K 31/675; A61K 31/436; A61P 35/00

(52) U.S. Cl. 514/80; 514/291

(57) ABSTRACT

The instant invention provides a method of treating a cancer selected from the group consisting of non-small cell lung cancer and breast cancer with an mTOR inhibitor and an αvβ3 integrin antagonist, wherein the mTOR inhibitor is ridaforolimus, everolimus, temsirolimus or a combination thereof.

### BACKGROUND OF THE INVENTION

The phosphatidylinositol-3-kinase (PI3K) signaling pathway is important for the growth and survival of cancer cells in many different types of human malignancy. See, Granville C A et al, “Handicapping the Race to Develop Inhibitors of the Phosphoinositide 4-Kinase/Akt/Mammalian Target of Rapamycin Pathway,” Clin Cancer Res, 2006; 12(3) 679-89. This pathway receives upstream input from ligand-receptor interactions, such as the epidermal growth factor receptor and insulin-like growth factor receptor, and signals through downstream effectors, such as the mammalian target of rapamycin (mTOR). mTOR is a critical downstream effector molecule that regulates the production of proteins critical for cell cycle progression and many other important cellular growth processes. See, Abraham R T and Gibbons, J J, “The mammalian target of rapamycin signaling pathway: twists and turns in the road to cancer therapy.” Clin Cancer Res, 2007; 13(11) 3109-14.

Dysregulation of the PI3 kinase axis is common in human cancer due to overactive growth factor receptor signaling, activating mutations of PI3K, loss of function of the PTEN tumor suppressor, and several other mechanisms that result in activation of mTOR kinase activity. Clinically, successful pharmacological inhibition of the PI3K axis has focused on the upstream growth factor receptors and the downstream effectors of PI3 kinase, such as mTOR. There is now substantial clinical evidence showing that mTOR inhibitors can provide clinical benefit to patients with advanced malignancies.

Integrins are heterodimeric receptors that play pivotal roles in diverse cellular processes, including cell migration, proliferation, and attachment. Tumor cells of several types of cancer, including melanoma, breast cancer, prostate cancer, colon cancer and glioma, express αvβ3 integrin; this expression has been shown to be associated with progression and metastasis in melanoma, breast cancer and prostate cancer. See Xiaoping Duan, et al., “ Association of integrin expression with the metastatic potent and migratory and chemotactic ability of human osteosarcoma cells,” Clinical & Experimental Metastasis (2004) 21:747-753. Integrin inhibition has shown potent anti-cancer effects in preclinical studies, and could have potential for clinical development.

### SUMMARY OF THE INVENTION

The instant invention provides a method of treating a cancer selected from the group consisting of non-small cell lung cancer and breast cancer with an mTOR inhibitor and an αvβ3 integrin antagonist, wherein the mTOR inhibitor is ridaforolimus, everolimus, temsirolimus, a rapamycin-analog or a combination thereof and the αvβ3 integrin antagonist is Compound A.

### BRIEF DESCRIPTION OF THE DRAWINGS

FIG. 1 ITGAV was identified in an siRNA screen for inducers or inhibitors of the ridaforolimus induced activation of Akt. A whole genome siRNA screen was performed in HT1080 cells in the presence of ridaforolimus. A mesoscale assay was used to determine the levels of phospho- and total Akt after siRNA transfection. The top 20 inducers and inhibitors of phospho-Akt are shown.

FIG. 2 Inhibition of integrin alpha V inhibits the ridaforolimus induced feedback loop on Akt. ITGAV knockdown in HT1080 cells with siRNA inhibits ridaforolimus induced activation of Akt (A). HT1080 (B) or MCF7 cells (C) were treated with 10 nM ridaforolimus or 10 μM Compound A or the combination of the two treatments overnight. Cells were then lysed and the levels of phospho-Akt and total Akt were detected by Western blot.

FIG. 3: Ridaforolimus & MK-0429 are synergistic in inhibiting the growth cancer cell lines. A549 (A), MCF7 (B) and H1703 (C) cells were treated with an eight by eight matrix of ridaforomilus and Compound A. After 72 hrs cell viability was measured using Vialight (Lonza). Highest Single Agent (HSA) analysis was performed to determine if the combination is synergistic. VHSA values <0 are antagonistic, =0 are additive, >0 are synergistic, ≧0.1 truly synergistic, ≧0.2 strongly synergistic.

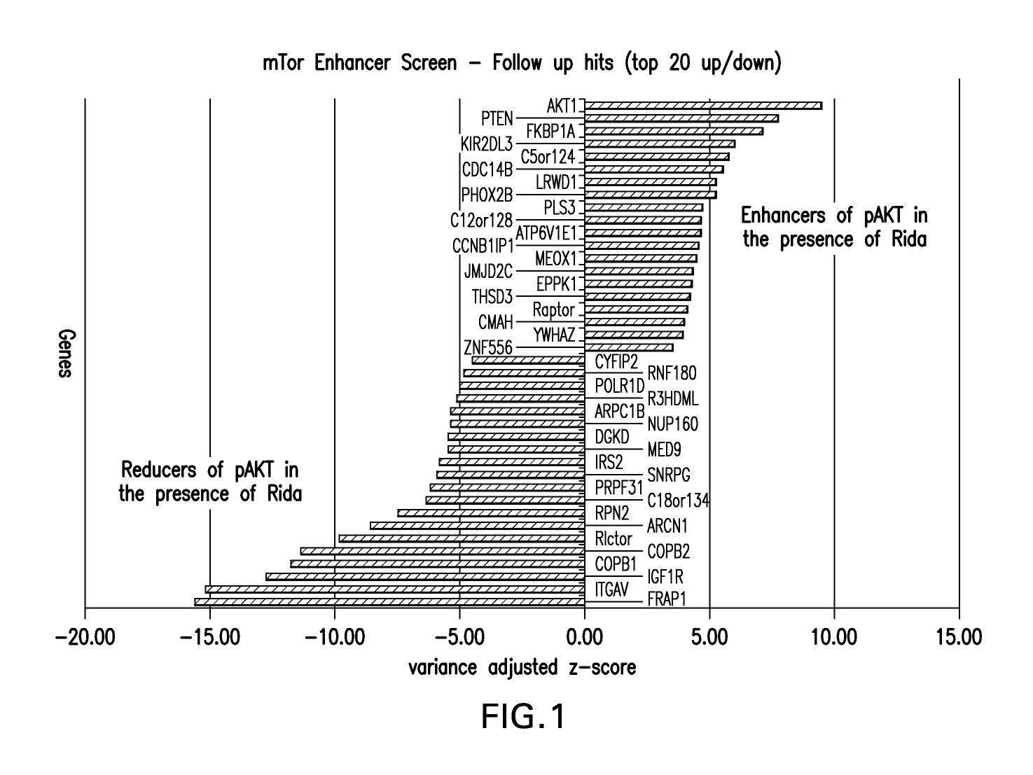

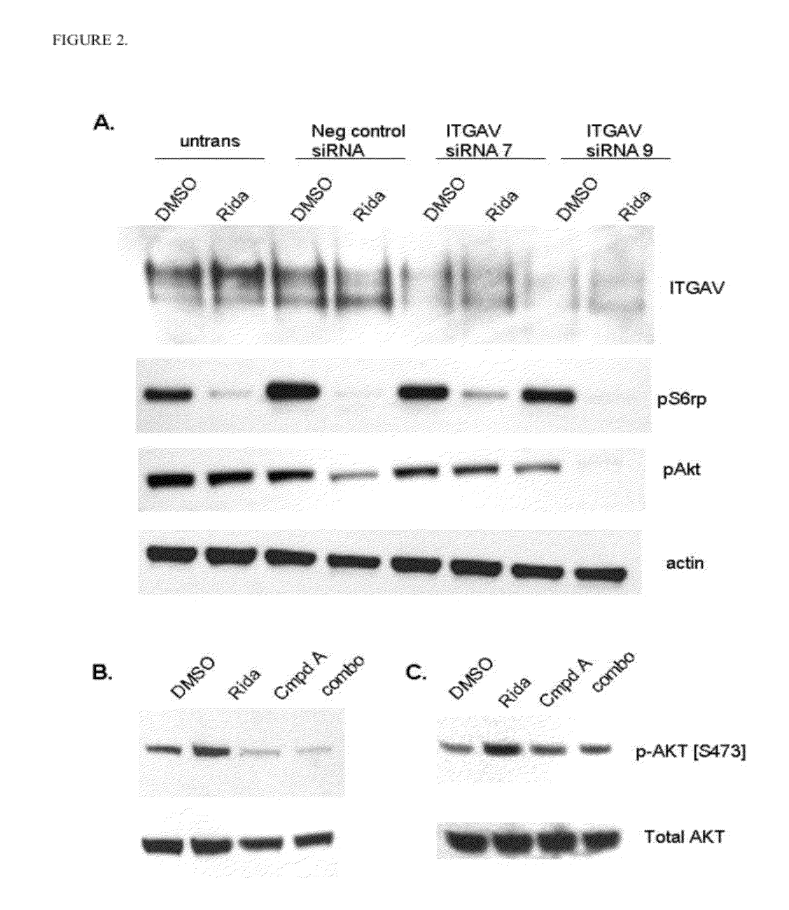

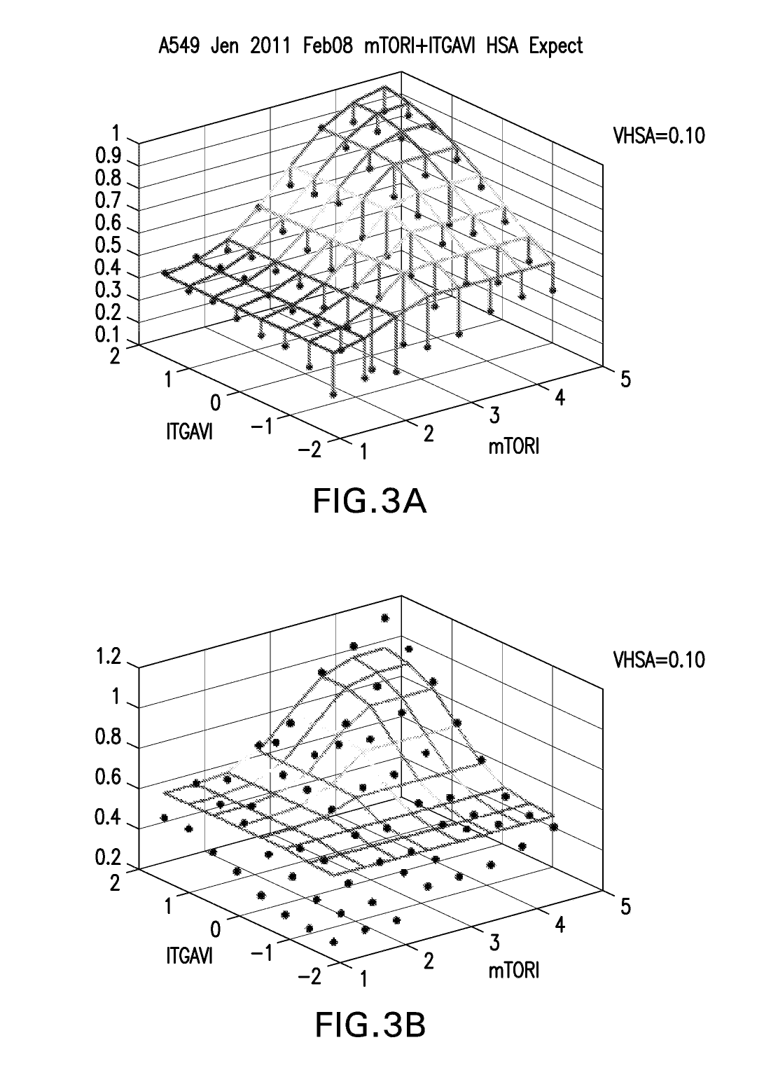

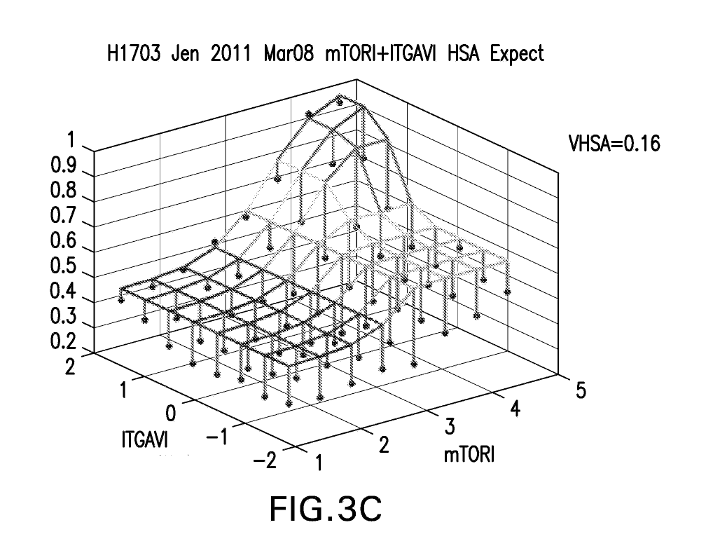

### DETAILED DESCRIPTION OF THE INVENTION

The combination of mTOR and αvβ3 integrin antagonists may provide a synergistic effect by inhibiting both upstream and downstream molecular targets in the PI3K pathway. The inhibition of mTOR can lead to the activation of a feedback loop that activates the Akt oncogene, which manifests as increased levels of phospho-Akt in tumor cells in vitro and from tumor biopsies taken from patients treated with mTOR inhibitors. See, Sun, S-Y et al., “Priority Report: Activation of Akt and eIF4E survival pathways by rapamycin-mediated mammalian target of rapamycin inhibition,” Cancer Res 2005; 65(16): 7052-58, and Gardner, H et al., “Biomarker analysis of a phase II double-blind randomized trial of daily oral RAD001 (everolimus) plus letrozole or placebo plus letrozole as neoadjuvant therapy for patients with estrogen receptor positive breast cancer,” San Antonio Breast Cancer Symposium. San Antonio, Tex., Dec. 13-16, 2007. Abstract 2006. Inhibition of αvβ3 integrin can block the positive feedback loop on Akt and may be more efficacious than mTOR inhibitor monotherapy.

As a result, preclinical studies have shown that the combination of αvβ3 integrin antagonists and mTOR inhibitors leads to additive or synergistic anti-tumor activity in vitro; the present inventors have found that synergistically excellent anticancer activity can be achieved by using an mTOR inhibitor or a pharmaceutically acceptable salt thereof in combination with an αvβ3 integrin antagonist, wherein the mTOR inhibitor is ridaforolimus, everolimus, temsirolimus, a rapamycin-analog or a combination thereof, and the αvβ3 integrin antagonist is Compound A. The invention is especially useful in the treatment of a cancer selected from the group consisting of non-small cell lung cancer and breast cancer. However, the instant invention could prove useful in the treatment of various other cancers, such as brain cancer, cervicocerebral cancer, colorectal cancer, soft tissue or bone sarcomas, endometrial cancer, esophageal cancer, thyroid cancer, small cell lung cancer, lung cancer, stomach cancer, gallbladder/bile duct cancer, liver cancer, pancreatic cancer, ovarian cancer, choriocarcinoma, uterus body cancer, uterocervical cancer, renal pelvis/ureter cancer, bladder cancer, prostate cancer, penis cancer, testicles cancer, fetal cancer, Wilms' cancer, skin cancer, malignant melanoma, neuroblastoma, osteosarcoma, Ewing's tumor, soft part sarcoma, acute leukemia, chronic lymphatic leukemia, chronic myelocytic leukemia and Hodgkin's lymphoma.

Accordingly, the instant invention relates to a method of treating a cancer selected from the group consisting of non-small cell lung cancer and breast cancer, with an mTOR inhibitor and an αvβ3 integrin antagonist, wherein the mTOR inhibitor is ridaforolimus, everolimus, temsirolimus, a rapamycin-analog or a combination thereof, and the αvβ3 integrin antagonist is Compound A.

In an embodiment of the invention, the mTOR inhibitor is ridaforolimus.

In another embodiment of the invention, the αvβ3 integrin antagonist is Compound A.

In another embodiment of the invention, the mTOR inhibitor is ridaforolimus and the αvβ3 integrin antagonist is Compound A.

In another embodiment of the invention, the mTOR inhibitor is administered in a dose between 10 mg and 40 mg. In a class of the invention, the αvβ3 integrin antagonist is administered in doses from about 200 mg to 1600 mg per day.

The mTOR inhibitor and the αvβ3 integrin antagonist can be prepared for simultaneous, separate or successive administration.

Reference to the preferred embodiments set forth above is meant to include all combinations of particular and preferred groups unless stated otherwise. The meanings of the terms used in this description are described below, and the invention is described in more detail hereinunder.

The term “simultaneous” as referred to in this description means that the pharmaceutical preparations of the invention are administered simultaneously in time.

The term “separate” as referred to in this description means that the pharmaceutical preparations of the invention are administered at different times during the course of a common treatment schedule.

The term “successive” as referred to in this description means that administration of one pharmaceutical preparation is followed by administration of the other pharmaceutical preparation; after administration of one pharmaceutical preparation, the second pharmaceutical preparation can be administered substantially immediately after the first pharmaceutical preparation, or the second pharmaceutical preparation can be administered after an effective time period after the first pharmaceutical preparation; and the effective time period is the amount of time given for realization of maximum benefit from the administration of the first pharmaceutical preparation.

The term “cancer” as referred to in this description includes various sarcoma and carcinoma and includes solid cancer and hematopoietic cancer. The solid cancer as referred to herein includes, for example, brain cancer, cervicocerebral cancer, esophageal cancer, thyroid cancer, small cell lung cancer, non-small cell lung cancer, breast cancer, endometrial cancer, lung cancer, stomach cancer, gallbladder/bile duct cancer, liver cancer, pancreatic cancer, colon cancer, rectal cancer, ovarian cancer, choriocarcinoma, uterus body cancer, uterocervical cancer, renal pelvis/ureter cancer, bladder cancer, prostate cancer, penis cancer, testicles cancer, fetal cancer, Wilms' tumor, skin cancer, malignant melanoma, neuroblastoma, osteosarcoma, Ewing's tumor, soft part sarcoma. On the other hand, the hematopoietic cancer includes, for example, acute leukemia, chronic lymphatic leukemia, chronic myelocytic leukemia, polycythemia vera, malignant lymphoma, multiple myeloma, Hodgkin's lymphoma, non-Hodgkin's lymphoma.

The term “treatment of cancer” as referred to in this description means that an anticancer agent is administered to a cancer case so as to inhibit the growth of the cancer cells in the case. Preferably, the treatment results in cancer growth regression, or that is, it reduces the size of a detectable cancer. More preferably, the treatment results in complete disappearance of cancer.

mTOR Inhibitors

The mTOR inhibitors in current clinical development are structural analogs of rapamycin. The mTOR inhibitors of the instant invention include ridaforolimus, temsirolimus, everolimus, a rapamycin-analog and combinations thereof.

Ridaforolimus, also known as AP 23573, MK-8669, Rida and deforolimus, is a unique, non-prodrug analog of rapmycin that has antiproliferative activity in a broad range of human tumor cell lines in vitro and in murine tumor xenograft models utilizing human tumor cell lines. Ridaforolimus has been administered to patients with advanced cancer and is currently in clinical development for various advanced malignancies, including studies in patients with advanced soft tissue or bone sarcomas. Thus far, these trials have demonstrated that ridaforolimus is generally well-tolerated with a predictable and manageable adverse even profile, and possess anti-tumor activity in a broad range of cancers. A description and preparation of ridaforolimus is described in U.S. Pat. No. 7,091,213 to Ariad Gene Therapeutics, Inc., which is hereby incorporated by reference in its entirety.

Temsirolimus, also known as Torisel®, is currently marketed for the treatment of renal cell carcinoma. A description and preparation of temsirolimus is described in U.S. Pat. No. 5,362,718 to American Home Products Corporation, which is hereby incorporated by reference in its entirety.

Everolimus, also known as Certican® or RAD001, marketed by Novartis, has greater stability and enhanced solubility in organic solvents, as well as more favorable pharmokinetics with fewer side effects than rapamycin (sirolimus). Everolimus has been used in conjunction with microemulsion cyclosporin (Neoral®, Novartis) to increase the efficacy of the immunosuppressive regime.

The mTOR inhibitors of the instant invention may also exist as various crystals, amorphous substances, pharmaceutically acceptable salts, hydrates and solvates. Further, the mTOR inhibitors of the instant invention may be provided as prodrugs. In general, such prodrugs are functional derivatives of the mTOR inhibitors of the instant invention that can be readily converted into compounds that are needed by living bodies. Accordingly, in the method of treatment of various cancers in the invention, the term “administration” includes not only the administration of a specific compound but also the administration of a compound which, after administered to patients, can be converted into the specific compound in the living bodies. Conventional methods for selection and production of suitable prodrug derivatives are described, for example, in “Design of Prodrugs”, ed. H. Bundgaard, Elsevier, 1985, which is referred to herein and is entirely incorporated herein as a part of the present description. Metabolites of the compound may include active compounds that are produced by putting the compound in a biological environment, and are within the scope of the compound in the invention.

αvβ3 Integrin Antagonists

The αvβ3 integrin antagonists of the instant invention have been described in U.S. Pat. Nos. 6,017,926; 6,297,249 and 6,472,403, which are incorporated by reference herein in their entirety.

U.S. Pat. No. 6,017,926 (issued Jan. 25, 2000) discloses compounds of structural formula I:

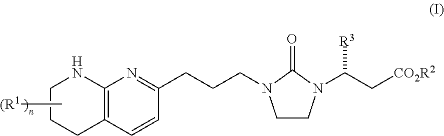

Wherein each R¹ is independently selelcted from the group consisting of hydrogen, C₁₋₄ alkyl and cyclopropyl; or two R¹ substituents, when on the same carbon atom, are taken together with the carbon atom to which they are attached to form a spirocyclopropyl group;
R² is hydrogen or C₁₋₄ alkyl;
R³ is mono-or di-substituted quinolinyl, pyridinyl or pyrimidinyl; wherein the substituents are each independently selected from the group consisting of hydrogen, halo, phenyl, C₁₋₄ alkyl, C₃₋₆ cycloalkyl, C₁₋₃alkoxy, amino, C₁₋₃alkylamino, di(C₁₋₃alkylamino), hydroxyl, cyano, trifluoromethyl, trifluoroethyl, trifluoromethoxy and trifluoroethoxy.

In an emobidment of the invention, the αvβ3 integrin antagonist of the instant invention is

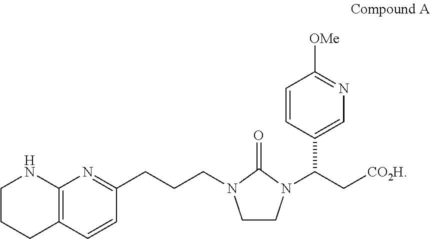

Compound A is an antagonist of the integrin αvβ3 receptor and is useful for inhibiting bone resorption, restenosis, angiogenesis, diabetic retinopathy, macular degeneration, inflammatory arthritis, cancer, and metastatic tumor growth. Compound A is also known as MK-0429 or Cmpd A. Novel processes and intermediates for the preparation of Compound A are disclosed in U.S. Pat. Nos. 6,262,268; 6,407,241; 6,423,845; 6,706,885; 6,646,130; and 6,914,144, and in Nobuyoski Yasuda, et a;., “An Efficient Synthesis of an αvβ3 Antagonist,” J. Org. Chem. 2004, 69, 1959-1966, which are hereby incorporated by reference in their entirety. Hydroxylated metabolites of Compound A are disclosed in U.S. Pat. No. 6,426,353, which is hereby incorporated by reference in its entirety. Crystalline hydrates of Compound A are disclosed in U.S. Pat. No. 6,509,347, which is hereby incorporated by reference in its entirety.

The compounds of the present invention may have asymmetric centers, chiral axes, and chiral planes (as described in: E. L. Eliel and S. H. Wilen, Stereochemistry of Carbon Compounds, John Wiley & Sons, New York, 1994, pages 1119-1190), and occur as racemates, racemic mixtures, and as individual diastereomers, with all possible isomers and mixtures thereof, including optical isomers, all such stereoisomers being included in the present invention. In addition, the compounds disclosed herein may exist as tautomers and both tautomeric forms are intended to be encompassed by the scope of the invention, even though only one tautomeric structure is depicted.

In the compounds of generic Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (1H) and deuterium (2H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

When any variable (e.g. R¹) occurs more than one time in any constituent, its definition on each occurrence is independent at every other occurrence. Also, combinations of substituents and variables are permissible only if such combinations result in stable compounds. Lines drawn into the ring systems from substituents represent that the indicated bond may be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. The phrase “optionally substituted with one or more substituents” should be taken to be equivalent to the phrase “optionally substituted with at least one substituent” and in such cases another embodiment will have from zero to three substituents.

As used herein, “alkyl” is intended to include both branched and straight-chain saturated aliphatic hydrocarbon groups having the specified number of carbon atoms. For example, C₁-C₁₀, as in “C₁-C₁₀ alkyl” is defined to include groups having 1, 2, 3, 4, 5, 6, 7, 8, 9 or 10 carbons in a linear or branched arrangement. For example, “C₁-C₁₀ alkyl” specifically includes methyl, ethyl, n-propyl, i-propyl, n-butyl, t-butyl, i-butyl, pentyl, hexyl, heptyl, octyl, nonyl, decyl, and so on. The term “cycloalkyl” means a monocyclic saturated aliphatic hydrocarbon group having the specified number of carbon atoms. For example, “cycloalkyl” includes cyclopropyl, methyl-cyclopropyl, 2,2-dimethyl-cyclobutyl, 2-ethyl-cyclopentyl, cyclohexyl, and so on. In an embodiment of the invention the term “cycloalkyl” includes the groups described immediately above and further includes monocyclic unsaturated aliphatic hydrocarbon groups. For example, “cycloalkyl” as defined in this embodiment includes cyclopropyl, methyl-cyclopropyl, 2,2-dimethyl-cyclobutyl, 2-ethyl-cyclopentyl, cyclohexyl, cyclopentenyl, cyclobutenyl and so on.

The term “haloalkyl” means an alkyl radical as defined above, unless otherwise specified, that is substituted with one to five, preferably one to three halogen. Representative examples include, but are not limited to trifluoromethyl, dichloroethyl, and the like.

“Alkoxy” represents either a cyclic or non-cyclic alkyl group of indicated number of carbon atoms attached through an oxygen bridge. “Alkoxy” therefore encompasses the definitions of alkyl and cycloalkyl above.

### Dosing and Routes of Administration

With regard to the mTOR inhibitors and αvβ3 integrin antagonists of the invention, various preparation forms can be selected, and examples thereof include oral preparations such as tablets, capsules, powders, granules or liquids, or sterilized liquid parenteral preparations such as solutions or suspensions, suppositories, ointments and the like. The mTOR inhibitors are available as pharmaceutically acceptable salts. The mTOR inhibitors and αvβ3 integrin antagonists of the invention are prepared with pharmaceutically acceptable carriers or diluents.

The term “pharmaceutically acceptable salt” as referred to in this description means ordinary, pharmaceutically acceptable salt. For example, when the compound has a hydroxyl group, or an acidic group such as a carboxyl group and a tetrazolyl group, then it may form a base-addition salt at the hydroxyl group or the acidic group; or when the compound has an amino group or a basic heterocyclic group, then it may form an acid-addition salt at the amino group or the basic heterocyclic group.

The base-addition salts include, for example, alkali metal salts such as sodium salts, potassium salts; alkaline earth metal salts such as calcium salts, magnesium salts; ammonium salts; and organic amine salts such as trimethylamine salts, triethylamine salts, dicyclohexylamine salts, ethanolamine salts, diethanolamine salts, triethanolamine salts, procaine salts, N,N′-dibenzylethylenediamine salts.

The acid-addition salts include, for example, inorganic acid salts such as hydrochlorides, sulfates, nitrates, phosphates, perchlorates; organic acid salts such as maleates, fumarates, tartrates, citrates, ascorbates, trifluoroacetates; and sulfonates such as methanesulfonates, isethionates, benzenesulfonates, p-toluenesulfonates.

The term “pharmaceutically acceptable carrier or diluent” refers to excipients [e.g., fats, beeswax, semi-solid and liquid polyols, natural or hydrogenated oils, etc.]; water (e.g., distilled water, particularly distilled water for injection, etc.), physiological saline, alcohol (e.g., ethanol), glycerol, polyols, aqueous glucose solution, mannitol, plant oils, etc.); additives [e.g., extending agent, disintegrating agent, binder, lubricant, wetting agent, stabilizer, emulsifier, dispersant, preservative, sweetener, colorant, seasoning agent or aromatizer, concentrating agent, diluent, buffer substance, solvent or solubilizing agent, chemical for achieving storage effect, salt for modifying osmotic pressure, coating agent or antioxidant], and the like.

Solid preparations can be prepared in the forms of tablet, capsule, granule and powder without any additives, or prepared using appropriate carriers (additives). Examples of such carriers (additives) may include saccharides such as lactose or glucose; starch of corn, wheat or rice; fatty acids such as stearic acid; inorganic salts such as magnesium metasilicate aluminate or anhydrous calcium phosphate; synthetic polymers such as polyvinylpyrrolidone or polyalkylene glycol; alcohols such as stearyl alcohol or benzyl alcohol; synthetic cellulose derivatives such as methylcellulose, carboxymethylcellulose, ethylcellulose or hydroxypropylmethylcellulose; and other conventionally used additives such as gelatin, talc, plant oil and gum arabic.

These solid preparations such as tablets, capsules, granules and powders may generally contain, for example, 0.1 to 100% by weight, and preferably 5 to 98% by weight, of the mTOR inhibitor, based on the total weight of each preparation.

Liquid preparations are produced in the forms of suspension, syrup, injection and drip infusion (intravenous fluid) using appropriate additives that are conventionally used in liquid preparations, such as water, alcohol or a plant-derived oil such as soybean oil, peanut oil and sesame oil.

In particular, when the preparation is administered parenterally in a form of intramuscular injection, intravenous injection or subcutaneous injection, appropriate solvent or diluent may be exemplified by distilled water for injection, an aqueous solution of lidocaine hydrochloride (for intramuscular injection), physiological saline, aqueous glucose solution, ethanol, polyethylene glycol, propylene glycol, liquid for intravenous injection (e.g., an aqueous solution of citric acid, sodium citrate and the like) or an electrolytic solution (for intravenous drip infusion and intravenous injection), or a mixed solution thereof.

Such injection may be in a form of a preliminarily dissolved solution, or in a form of powder per se or powder associated with a suitable carrier (additive) which is dissolved at the time of use. The injection liquid may contain, for example, 0.1 to 10% by weight of an active ingredient based on the total weight of each preparation.

Liquid preparations such as suspension or syrup for oral administration may contain, for example, 0.1 to 10% by weight of an active ingredient based on the total weight of each preparation.

Each preparation in the invention can be prepared by a person having ordinary skill in the art according to conventional methods or common techniques. For example, a preparation can be carried out, if the preparation is an oral preparation, for example, by mixing an appropriate amount of the compound of the invention with an appropriate amount of lactose and filling this mixture into hard gelatin capsules which are suitable for oral administration. On the other hand, preparation can be carried out, if the preparation containing the compound of the invention is an injection, for example, by mixing an appropriate amount of the compound of the invention with an appropriate amount of 0.9% physiological saline and filling this mixture in vials for injection.

The components of this invention may be administered to mammals, including humans, either alone or, in combination with pharmaceutically acceptable carriers, excipients or diluents, in a pharmaceutical composition, according to standard pharmaceutical practice. The components can be administered orally or parenterally, including the intravenous, intramuscular, intraperitoneal, subcutaneous, rectal and topical routes of administration.

Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved. Further information about suitable dosages is provided below.

The term “administration” and variants thereof (e.g., “administering” a compound) in reference to a component of the invention means introducing the component or a prodrug of the component into the system of the animal in need of treatment. When a component of the invention or prodrug thereof is provided in combination with one or more other active agents (e.g., the mTOR inhibitor), “administration” and its variants are each understood to include concurrent and sequential introduction of the component or prodrug thereof and other agents.

As used herein, the term “composition” is intended to encompass a product comprising the specified ingredients in the specified amounts, as well as any product which results, directly or indirectly, from combination of the specified ingredients in the specified amounts.

The term “therapeutically effective amount” as used herein means that amount of active compound or pharmaceutical agent that elicits the biological or medicinal response in a tissue, system, animal or human that is being sought by a researcher, veterinarian, medical doctor or other clinician.

A suitable amount of an mTOR inhibitor is administered to a patient undergoing treatment for cancer. In an embodiment, the mTOR inhibitor is administered in doses from about 10 mg-40 mg per day. In an embodiment of the invention, the mTOR inhibitor is administered in a dose of 10 mg per day. In another embodiment of the invention, the mTOR inhibitor is administered in a dose of 20 mg per day. In another embodiment of the invention, the mTOR inhibitor is administered in a dose of 30 mg per day. In another embodiment of the invention, the mTOR inhibitor is administered in a dose of 40 mg per day.

In an embodiment of the invention, the mTOR inhibitor can be administered 5 times per week. For example, ridaforolimus is started on Day 1, and continued at the specified dosing level for five consecutive days, followed by two days of no ridaforolimus treatment. Ridaforolimus is then continued on this daily×5 schedule each week.

A suitable amount of an αvβ3 integrin antagonist is administered to a patient undergoing treatment for cancer. In an embodiment, the αvβ3 integrin antagonist is administered in doses from about 200 mg to 1600 mg per day. In an embodiment of the invention, the αvβ3 integrin antagonist will be dosed BID daily.

In a broad embodiment, the treatment of the present invention involves the combined administration of an αvβ3 integrin antagonist and an mTOR inhibitor. The combined administration includes co administration, using separate formulations or a single pharmaceutical formulation, and consecutive administration in either order, wherein preferably there is a time period while both (or all) active agents simultaneously exert their biological activities. Preparation and dosing schedules for such chemotherapeutic agents may be used according to manufacturers' instructions or as determined empirically by the skilled practitioner. Preparation and dosing schedules for chemotherapy are also described in Chemotherapy Service Ed., M. C. Perry, Williams & Wilkins, Baltimore, Md. (1992). The mTOR inhibitor may precede, or follow administration of the αvβ3 integrin antagonist or may be given simultaneously therewith. The clinical dosing of therapeutic combination of the present invention are likely to be limited by the extent of adverse reactions.

### Additional Indications

In addition to the treatment of non-small cell lung cancer, breast cancer, colorectal cancer, soft tissue or bone sarcomas and endometrial cancer, the mTOR inhibitor and αvβ3 integrin antagonist combination may also be useful for the treatment of the following cancers: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma,; and Adrenal glands: neuroblastoma. Thus, the term “cancerous cell” as provided herein, includes a cell afflicted by any one of the above-identified conditions.

The mTOR inhibitor and αvβ3 integrin antagonist combination of the invention may also be useful in treating the following disease states: keloids and psoriasis.

Further included within the scope of the invention is a method of treating or preventing a disease in which angiogenesis is implicated, which is comprised of administering to a mammal in need of such treatment a therapeutically effective amount of the combination of the present invention. Ocular neovascular diseases are an example of conditions where much of the resulting tissue damage can be attributed to aberrant infiltration of blood vessels in the eye (see WO 00/30651, published 2 Jun. 2000). The undesireable infiltration can be triggered by ischemic retinopathy, such as that resulting from diabetic retinopathy, retinopathy of prematurity, retinal vein occlusions, etc., or by degenerative diseases, such as the choroidal neovascularization observed in age-related macular degeneration. Inhibiting the growth of blood vessels by administration of the present compounds would therefore prevent the infiltration of blood vessels and prevent or treat diseases where angiogenesis is implicated, such as ocular diseases like retinal vascularization, diabetic retinopathy, age-related macular degeneration, and the like.

Further included within the scope of the invention is a method of treating or preventing a non-malignant disease in which angiogenesis is implicated, including but not limited to: ocular diseases (such as, retinal vascularization, diabetic retinopathy and age-related macular degeneration), atherosclerosis, arthritis, psoriasis, obesity and Alzheimer's disease (Dredge et al., Expert Opin. Biol. Ther. (2002) 2(8):953-966). In another embodiment, a method of treating or preventing a disease in which angiogenesis is implicated includes: ocular diseases (such as, retinal vascularization, diabetic retinopathy and age-related macular degeneration), atherosclerosis, arthritis and psoriasis.

Further included within the scope of the invention is a method of treating hyperproliferative disorders such as restenosis, inflammation, autoimmune diseases and allergy/asthma.

Further included within the scope of the instant invention is the use of the instant combination to coat stents and therefore the use of the instant compounds on coated stents for the treatment and/or prevention of restenosis (WO03/032809).

Further included within the scope of the instant invention is the use of the instant combination for the treatment and/or prevention of osteoarthritis (WO03/035048).

Further included within the scope of the invention is a method of treating hypoinsulinism.

Exemplifying the invention is the use of the mTOR inhibitor and αvβ3 integrin antagonist combination described above in the preparation of a medicament for the treatment and/or prevention of non-small cell lung cancer, breast cancer, colorectal cancer, soft tissue or bone sarcomas and endometrial cancer.

### Additional Anti-Cancer Agents

The mTOR inhibitor and αvβ3 integrin antagonist combination of the instant invention is also useful in combination with additional therapeutic, chemotherapeutic and anti-cancer agents. Further combinations of the mTOR inhibitor and αvβ3 integrin antagonist combination of the instant invention with therapeutic, chemotherapeutic and anti-cancer agents are within the scope of the invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Such additional agents include the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, HIV protease inhibitors, reverse transcriptase inhibitors, inhibitors of cell proliferation and survival signaling, bisphosphonates, aromatase inhibitors, siRNA therapeutics, γ-secretase inhibitors, agents that interfere with receptor tyrosine kinases (RTKs) and agents that interfere with cell cycle checkpoints. The mTOR inhibitor and αvβ3 integrin antagonist combination of the instant invention may be particularly useful when co-administered with radiation therapy.

“Estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-hydrazone, and SH646.

“Androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

“Retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, α-difluoromethylornithine, ILX23-7553, trans-N-(4′-hydroxyphenyl) retinamide, and N-4-carboxyphenyl retinamide.

“Cytotoxic/cytostatic agents” refer to compounds which cause cell death or inhibit cell proliferation primarily by interfering directly with the cell's functioning or inhibit or interfere with cell myosis, including alkylating agents, tumor necrosis factors, intercalators, hypoxia activatable compounds, microtubule inhibitors/microtubule-stabilizing agents, inhibitors of mitotic kinesins, histone deacetylase inhibitors, inhibitors of kinases involved in mitotic progression, inhibitors of kinases involved in growth factor and cytokine signal transduction pathways, antimetabolites, biological response modifiers, hormonal/anti-hormonal therapeutic agents, haematopoietic growth factors, monoclonal antibody targeted therapeutic agents, topoisomerase inhibitors, proteosome inhibitors, ubiquitin ligase inhibitors, and aurora kinase inhibitors.

Examples of cytotoxic/cytostatic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)[bis[diamine(chloro)platinum (II)]tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxycarminomycin, annamycin, galarubicin, elinafide, MEN10755, 4-demethoxy-3-deamino-3-aziridinyl-4-methylsulphonyl-daunorubicin (see WO 00/50032), Raf kinase inhibitors (such as Bay43-9006) and additional mTOR inhibitors.

An example of a hypoxia activatable compound is tirapazamine.

Examples of proteosome inhibitors include but are not limited to lactacystin and MLN-341 (Velcade).

Examples of microtubule inhibitors/microtubule-stabilising agents include paclitaxel, vindesine sulfate, 3′,4′-didehydro-4′-deoxy-8′-norvincaleukoblastine, docetaxol, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797. In an embodiment the epothilones are not included in the microtubule inhibitors/microtubule-stabilising agents.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-k1]acridine-2-(6H) propanamine, 1-amino-9-ethyl-5-fluoro-2,3-dihydro-9-hydroxy-4-methyl-1H,12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a, 5aB, 8aa,9b)-9-[2-[N-[2-(dimethylamino)ethyl]-N-methylamino]ethyl]-5-[4-hydro0xy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c] quinolin-7-one, and dimesna.

Examples of inhibitors of mitotic kinesins, and in particular the human mitotic kinesin KSP, are described in Publications WO03/039460, WO03/050064, WO03/050122, WO03/049527, WO03/049679, WO03/049678, WO04/039774, WO03/079973, WO03/099211, WO03/105855, WO03/106417, WO04/037171, WO04/058148, WO04/058700, WO04/126699, WO05/018638, WO05/019206, WO05/019205, WO05/018547, WO05/017190, US2005/0176776. In an embodiment inhibitors of mitotic kinesins include, but are not limited to inhibitors of KSP, inhibitors of MKLP1, inhibitors of CENP-E, inhibitors of MCAK and inhibitors of Rab6-KIFL.

Examples of “histone deacetylase inhibitors” include, but are not limited to, SAHA, TSA, oxamflatin, PXD101, MG98 and scriptaid. Further reference to other histone deacetylase inhibitors may be found in the following manuscript; Miller, T. A. et al. J. Med. Chem. 46(24):5097-5116 (2003).

“Inhibitors of kinases involved in mitotic progression” include, but are not limited to, inhibitors of aurora kinase, inhibitors of Polo-like kinases (PLK; in particular inhibitors of PLK-1), inhibitors of bub-1 and inhibitors of bub-R1. An example of an “aurora kinase inhibitor” is VX-680.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N4-palmitoyl-1-B-D-arabino furanosyl cytosine, 3-aminopyridine-2-carboxaldehyde thiosemicarbazone and trastuzumab.

Examples of monoclonal antibody targeted therapeutic agents include those therapeutic agents which have cytotoxic agents or radioisotopes attached to a cancer cell specific or target cell specific monoclonal antibody. Examples include Bexxar.

“HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896), atorvastatin (LIPITOR®; see U.S. Pat. Nos. 5,273,995, 4,681,893, 5,489,691 and 5,342,952) and cerivastatin (also known as rivastatin and BAYCHOL®; see U.S. Pat. No. 5,177,080). The structural formulas of these and additional HMG-CoA reductase inhibitors that may be used in the instant methods are described at page 87 of M. Yalpani, “Cholesterol Lowering Drugs”, Chemistry & Industry, pp.

85-89 (5 February 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefor the use of such salts, esters, open-acid and lactone forms is included within the scope of this invention.

“Prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. No. 5,420,245, U.S. Pat. No. 5,523,430, U.S. Pat. No. 5,532,359, U.S. Pat. No. 5,510,510, U.S. Pat. No. 5,589,485, U.S. Pat. No. 5,602,098, European Patent Publ. 0 618 221, European Patent Publ. 0 675 112, European Patent Publ. 0 604 181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO 97/30053, WO 97/44350, WO 98/02436, and U.S. Pat. No. 5,532,359. For an example of the role of a prenyl-protein transferase inhibitor on angiogenesis see European J. of Cancer, Vol. 35, No. 9, pp. 1394-1401 (1999).

“Angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR1) and Flk-1/KDR (VEGFR2), inhibitors of epidermal-derived, fibroblast-derived, or platelet derived growth factors, MMP (matrix metalloprotease) inhibitors, integrin blockers, interferon-α, interleukin-12, pentosan polysulfate, cyclooxygenase inhibitors, including nonsteroidal anti-inflammatories (NSAIDs) like aspirin and ibuprofen as well as selective cyclooxy-genase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin, Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFIa]) (see Thrombosis Res. 101:329-354 (2001)). TAFIa inhibitors have been described in U.S. Ser. Nos. 60/310,927 (filed Aug. 8, 2001) and 60/349,925 (filed Jan. 18, 2002).

“Agents that interfere with cell cycle checkpoints” refer to compounds that inhibit protein kinases that transduce cell cycle checkpoint signals, thereby sensitizing the cancer cell to DNA damaging agents. Such agents include inhibitors of ATR, ATM, the CHK11 and CHK12 kinases and cdk and cdc kinase inhibitors and are specifically exemplified by 7-hydroxystaurosporin, flavopiridol, CYC202 (Cyclacel) and BMS-387032.

“Agents that interfere with receptor tyrosine kinases (RTKs)” refer to compounds that inhibit RTKs and therefore mechanisms involved in oncogenesis and tumor progression. Such agents include inhibitors of c-Kit, Eph, PDGF, Flt3 and c-Met. Further agents include inhibitors of RTKs as described by Bume-Jensen and Hunter, Nature, 411:355-365, 2001.

“Inhibitors of cell proliferation and survival signalling pathway” refer to compounds that inhibit signal transduction cascades downstream of cell surface receptors. Such agents include inhibitors of serine/threonine kinases (including but not limited to inhibitors of Akt such as described in WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734188, 60/652737, 60/670469), inhibitors of Raf kinase (for example BAY-43-9006), inhibitors of MEK (for example CI-1040 and PD-098059), inhibitors of mTOR (for example Wyeth CCI-779), and inhibitors of PI3K (for example LY294002).

Specific anti-IGF-1R antibodies include, but are not limited to, dalotuzumab, figitumumab, cixutumumab, SHC 717454, Roche R1507, EM164 or Amgen AMG479.

As described above, the combinations with NSAID's are directed to the use of NSAID's which are potent COX-2 inhibiting agents. For purposes of this specification an NSAID is potent if it possesses an IC₅₀ for the inhibition of COX-2 of 1 μM or less as measured by cell or microsomal assays.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC₅₀ for COX-2 over IC₅₀ for COX-1 evaluated by cell or microsomal assays. Such compounds include, but are not limited to those disclosed in U.S. Pat. No. 5,474,995, U.S. Pat. No. 5,861,419, U.S. Pat. No. 6,001,843, U.S. Pat. No. 6,020,343, U.S. Pat. No. 5,409,944, U.S. Pat. No. 5,436,265, U.S. Pat. No. 5,536,752, U.S. Pat. No. 5,550,142, U.S. Pat. No. 5,604,260, U.S. Pat. No. 5,698,584, U.S. Pat. No. 5,710,140, WO 94/15932, U.S. Pat. No. 5,344,991, U.S. Pat. No. 5,134,142, U.S. Pat. No. 5,380,738, U.S. Pat. No. 5,393,790, U.S. Pat. No. 5,466,823,U.S. Pat. No. 5,633,272 and U.S. Pat. No. 5,932,598, all of which are hereby incorporated by reference.

Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5-pyridinyl)pyridine; or a pharmaceutically acceptable salt thereof.

Compounds that have been described as specific inhibitors of COX-2 and are therefore useful in the present invention include, but are not limited to, the following: parecoxib, BEXTRA® and CELEBREX® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RPI4610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ3 integrin, to compounds which selectively antagonize, inhibit or counteract binding of a physiological ligand to the αvβ5 integrin, to compounds which antagonize, inhibit or counteract binding of a physiological ligand to both the αᵥβ3 integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁ and α₆β₄ integrins.

Some specific examples of tyrosine kinase inhibitors include N-(trifluoromethylphenyl)-5-methylisoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-k1]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N-4-chlorophenyl-4-(4-pyridylmethyl)-1-phthalazinamine, and EMD121974.

Combinations with compounds other than anti-cancer compounds are also encompassed in the instant methods. For example, combinations of the mTOR inhibitor and αvβ3 integrin antagonist combination of the instant invention with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999;274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice. (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid (disclosed in U.S. Ser. No. 09/782,856), and 2(R)-7-(3-(2-chloro-4-(4-fluorophenoxy)phenoxy)propoxy)-2-ethylchromane-2-carboxylic acid (disclosed in U.S. Ser. Nos. 60/235,708 and 60/244,697).

Another embodiment of the instant invention is the use of the presently disclosed compounds in combination with gene therapy for the treatment of cancer. For an overview of genetic strategies to treating cancer see Hall et al (Am. J. Hum. Genet. 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998;5(8):1105-13), and interferon gamma (J. Immunol. 2000;164:217-222).

The compounds of the instant invention may also be administered in combination with an inhibitor of inherent multidrug resistance (MDR), in particular MDR associated with high levels of expression of transporter proteins. Such MDR inhibitors include inhibitors of p-glycoprotein (P-gp), such as LY335979, XR9576, 0C144-093, R101922, VX853 and PSC833 (valspodar).

A compound of the present invention may be employed in conjunction with anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with other anti-emetic agents, especially neurokinin-1 receptor antagonists, 5HT3 receptor antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or others such as disclosed in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In another embodiment, conjunctive therapy with an anti-emesis agent selected from a neurokinin-1 receptor antagonist, a 5HT3 receptor antagonist and a corticosteroid is disclosed for the treatment or prevention of emesis that may result upon administration of the instant compounds.

Neurokinin-1 receptor antagonists of use in conjunction with the compounds of the present invention are fully described, for example, in U.S. Pat. Nos. 5,162,339, 5,232,929, 5,242,930, 5,373,003, 5,387,595, 5,459,270, 5,494,926, 5,496,833, 5,637,699, 5,719,147; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0 514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913,0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942 and 97/21702; and in British Patent Publication Nos. 2 266 529, 2 268 931, 2 269 170, 2 269 590, 2 271 774, 2 292 144, 2 293 168, 2 293 169, and 2 302 689. The preparation of such compounds is fully described in the aforementioned patents and publications, which are incorporated herein by reference.

In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

The mTOR inhibitor and αvβ3 integrin antagonist combination of the instant invention may also be administered with an agent useful in the treatment of anemia. Such an anemia treatment agent is, for example, a continuous eythropoiesis receptor activator (such as epoetin alfa).

The mTOR inhibitor and αvβ3 integrin antagonist combination of the instant invention may also be administered with an agent useful in the treatment of neutropenia. Such a neutropenia treatment agent is, for example, a hematopoietic growth factor which regulates the production and function of neutrophils such as a human granulocyte colony stimulating factor, (G-CSF). Examples of a G-CSF include filgrastim.

The mTOR inhibitor and αvβ3 integrin antagonist combination of the instant invention may also be administered with an immunologic-enhancing drug, such as levamisole, isoprinosine and Zadaxin.

The mTOR inhibitor and αvβ3 integrin antagonist combination of the instant invention may also be useful for treating or preventing cancer, including bone cancer, in combination with bisphosphonates (understood to include bisphosphonates, diphosphonates, bisphosphonic acids and diphosphonic acids). Examples of bisphosphonates include but are not limited to: etidronate (Didronel), pamidronate (Aredia), alendronate (Fosamax), risedronate (Actonel), zoledronate (Zometa), ibandronate (Boniva), incadronate or cimadronate, clodronate, EB-1053, minodronate, neridronate, piridronate and tiludronate including any and all pharmaceutically acceptable salts, derivatives, hydrates and mixtures thereof.

The mTOR inhibitor and αvβ3 integrin antagonist combination of the instant invention may also be useful for treating or preventing breast cancer in combination with aromatase inhibitors. Examples of aromatase inhibitors include but are not limited to: anastrozole, letrozole and exemestane.

The mTOR inhibitor and αvβ3 integrin antagonist combination of the instant invention may also be useful for treating or preventing cancer in combination with siRNA therapeutics.

The mTOR inhibitor and αvβ3 integrin antagonist combination of the instant invention may also be administered in combination with γ-secretase inhibitors and/or inhibitors of NOTCH signaling. Such inhibitors include compounds described in WO 01/90084, WO 02/30912, WO 01/70677, WO 03/013506, WO 02/36555, WO 03/093252, WO 03/093264, WO 03/093251, WO 03/093253, WO 2004/039800, WO 2004/039370, WO 2005/030731, WO 2005/014553, USSN 10/957,251, WO 2004/089911, WO 02/081435, WO 02/081433, WO 03/018543, WO 2004/031137, WO 2004/031139, WO 2004/031138, WO 2004/101538, WO 2004/101539 and WO 02/47671 (including LY-450139).

The mTOR inhibitor and αvβ3 integrin antagonist combination of the instant invention may also be useful for treating or preventing cancer in combination with inhibitors of Akt. Such inhibitors include compounds described in, but not limited to, the following publications: WO 02/083064, WO 02/083139, WO 02/083140, US 2004-0116432, WO 02/083138, US 2004-0102360, WO 03/086404, WO 03/086279, WO 03/086394, WO 03/084473, WO 03/086403, WO 2004/041162, WO 2004/096131, WO 2004/096129, WO 2004/096135, WO 2004/096130, WO 2005/100356, WO 2005/100344, US 2005/029941, US 2005/44294, US 2005/43361, 60/734188, 60/652737, 60/670469.

The mTOR inhibitor and αvβ3 integrin antagonist combination of the instant invention may also be useful for treating or preventing cancer in combination with PARP inhibitors.

Radiation therapy itself means an ordinary method in the field of treatment of cancer. For radiation therapy, employable are various radiations such as X-ray, γ-ray, neutron ray, electron beam, proton beam; and radiation sources. In a most popular radiation therapy, a linear accelerator is used for irradiation with external radiations, γ-ray.

The mTOR inhibitor and αvβ3 integrin antagonist combination of the instant invention may also be useful for treating cancer in further combination with the following therapeutic agents: abarelix (Plenaxis depot®); aldesleukin (Prokine®); Aldesleukin (Proleukin®); Alemtuzumabb (Campath®); alitretinoin (Panretin®); allopurinol (Zyloprim®); altretamine (Hexalen®); amifostine (Ethyol®); anastrozole (Arimidex®); arsenic trioxide (Trisenox®); asparaginase (Elspar®); azacitidine (Vidaza®); bevacuzimab (Avastin®); bexarotene capsules (Targretin®); bexarotene gel (Targretin®); bleomycin (Blenoxane®); bortezomib (Velcade®); busulfan intravenous (Busulfex®); busulfan oral (Myleran®); calusterone (Methosarb®); capecitabine (Xeloda®); carboplatin (Paraplatin®); carmustine (BCNU®, BiCNU®); carmustine (Gliadel®); carmustine with Polifeprosan 20 Implant (Gliadel Wafer®); celecoxib (Celebrex®); cetuximab (Erbitux®); chlorambucil (Leukeran®); cisplatin (Platinol®); cladribine (Leustatin®, 2-CdA®); clofarabine (Clolar®); cyclophosphamide (Cytoxan®, Neosar®); cyclophosphamide (Cytoxan Injection®); cyclophosphamide (Cytoxan Tablet®); cytarabine (Cytosar-U®); cytarabine liposomal (DepoCyt®); dacarbazine (DTIC-Dome®); dactinomycin, actinomycin D (Cosmegen®); Darbepoetin alfa (Aranesp®); daunorubicin liposomal (DanuoXome®); daunorubicin, daunomycin (Daunorubicin®); daunorubicin, daunomycin (Cerubidine®); Denileukin diftitox (Ontak®); dexrazoxane (Zinecard®); docetaxel (Taxotere®); doxorubicin (Adriamycin PFS®); doxorubicin (Adriamycin®, Rubex®); doxorubicin (Adriamycin PFS Injection®); doxorubicin liposomal (Doxil®); dromostanolone propionate (Dromostanolone ®); dromostanolone propionate (Masterone Injection®); Elliott's B Solution (Elliott's B Solution®); epirubicin (Ellence®); Epoetin alfa (epogen®); erlotinib (Tarceva®); estramustine (Emcyt®); etoposide phosphate (Etopophos®); etoposide, VP-16 (Vepesid®); exemestane (Aromasin®); Filgrastim (Neupogen®); floxuridine (intraarterial) (FUDR®); fludarabine (Fludara®); fluorouracil, 5-FU (Adrucil®); fulvestrant (Faslodex®); gefitinib (Iressa®); gemcitabine (Gemzar®); gemtuzumab ozogamicin (Mylotarg®); goserelin acetate (Zoladex Implant®); goserelin acetate (Zoladex®); histrelin acetate (Histrelin implant®); hydroxyurea (Hydrea®); Ibritumomab Tiuxetan (Zevalin®); idarubicin (Idamycin®); ifosfamide (IFEX®); imatinib mesylate (Gleevec®); interferon alfa 2a (Roferon A®); Interferon alfa-2b (Intron A®); irinotecan (Camptosar®); lenalidomide (Revlimid®); letrozole (Femara®); leucovorin (Wellcovorin®, Leucovorin®); Leuprolide Acetate (Eligard®); levamisole (Ergamisol®); lomustine, CCNU (CeeBU®); meclorethamine, nitrogen mustard (Mustargen®); megestrol acetate (Megace®); melphalan, L-PAM (Alkeran®); mercaptopurine, 6-MP (Purinethol®); mesna (Mesnex®); mesna (Mesnex tabs®); methotrexate (Methotrexate®); methoxsalen (Uvadex®); mitomycin C (Mutamycin®); mitotane (Lysodren®); mitoxantrone (Novantrone®); nandrolone phenpropionate (Durabolin-50®); nelarabine (Arranon®); Nofetumomab (Verluma®); Oprelvekin (Neumega®); oxaliplatin (Eloxatin®); paclitaxel (Paxene®); paclitaxel (Taxol®); paclitaxel protein-bound particles (Abraxane®); palifermin (Kepivance®); pamidronate (Aredia®); pegademase (Adagen (Pegademase Bovine)®); pegaspargase (Oncaspar®); Pegfilgrastim (Neulasta®); pemetrexed disodium (Alimta®); pentostatin (Nipent®); pipobroman (Vercyte®); plicamycin, mithramycin (Mithracin®); porfimer sodium (Photofrin®); procarbazine (Matulane®); quinacrine (Atabrine®); Rasburicase (Elitek®); Rituximab (Rituxan®); sargramostim (Leukine®); Sargramostim (Prokine®); sorafenib (Nexavar®); streptozocin (Zanosar®); sunitinib maleate (Sutent®); talc (Sclerosol®); tamoxifen (Nolvadex®); temozolomide (Temodar®); teniposide, VM-26 (Vumon®); testolactone (Teslac®); thioguanine, 6-TG (Thioguanine®); thiotepa (Thioplex®); topotecan (Hycamtin®); toremifene (Fareston®); Tositumomab (Bexxar®); Tositumomab/I-131 tositumomab (Bexxar®); Trastuzumab (Herceptin®); tretinoin, ATRA (Vesanoid®); Uracil Mustard (Uracil Mustard Capsules®); valrubicin (Valstar®); vinblastine (Velban®); vincristine (Oncovin®); vinorelbine (Navelbine®); and zoledronate (Zometa®).

All patents, publications and pending patent applications identified are hereby incorporated by reference.

The abbreviations used herein have the following tabulated meanings Abbreviations not tabulated below have their meanings as commonly used unless specifically stated otherwise.

| | |
|---|---|
| CH₂Cl₂ | Methylene chloride |
| Cu(OAc)₂ | Copper acetate |
| DCM | Dichloromethane |
| DIPEA | Diisopropanolamine |
| DMAP | 4-Dimethylaminopyridine |
| EDC | 1-ethyl-3-(3-dimethylaminopropyl) |
| | carbodiimide hydrochloride |
| Et₃N | Triethylamine |
| HCl | Hydrogen chloride |
| HOBt | N-hydroxybenzotriazole |
| H₂SO₄ | Sulfuric acide |
| MeOH | Methanol |
| MTBE | Methyl t-butyl ether |
| NaBH(OAc) | Sodium triacetoxyborohydride |
| NaCl | Sodium chloride |
| NaHCO₃ | Sodium bicarbonate |
| NaOAc | Sodium Acetate |
| NaOCl | Sodium hypochlorite |
| NaOH | Sodium hydroxide |
| NH₄Cl | Ammonium chloride |
| Pd(OAc)₂ | Palladium acetate |
| SiO₂ | Silicone dioxide |
| TsOH | Toluenesulfonic acid |

The mTOR inhibitors and αvβ3 integrin antagonist of the instant invention can be prepared according to the following general schemes, using appropriate materials, and are further exemplified by the subsequent specific examples. The specific anticancer agents illustrated in the examples are not, however, to be construed as forming the only genus that is considered as the invention. The illustrative Examples below, therefore, are not limited by the anticancer agents listed or by any particular substituents employed for illustrative purposes. Those skilled in the art will readily understand that known variations of the conditions and processes of the following preparative procedures can be used to prepare these compounds. All temperatures are degrees Celsius unless otherwise noted.

### METHODS OF SYNTHESIS

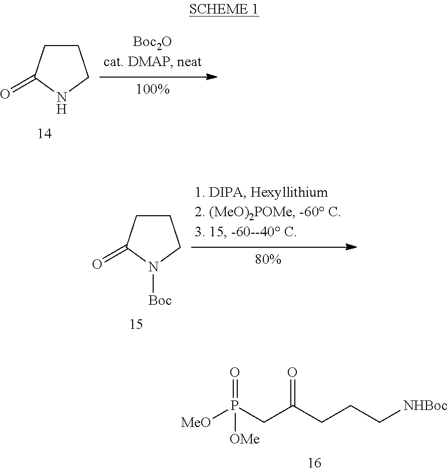

The preparation of 16 is summarized in Scheme 1. In the presence of a catalytic amount of DMAP, N-Boc-2-pyrrolidone (15) was prepared from 2-pyrrolidone (14) and di-tert-butyl dicarbonate neat in quantitative yield. The pyrrolidone ring of 15 was opened with the anion derived from dimethyl methylphosphonate to yield 16 in 80-85% isolated yield. (Flynn, D. L.; Zelle, R. E.; Grieco, P. A., J. Org. Chem., 1983, 48, 2424.)

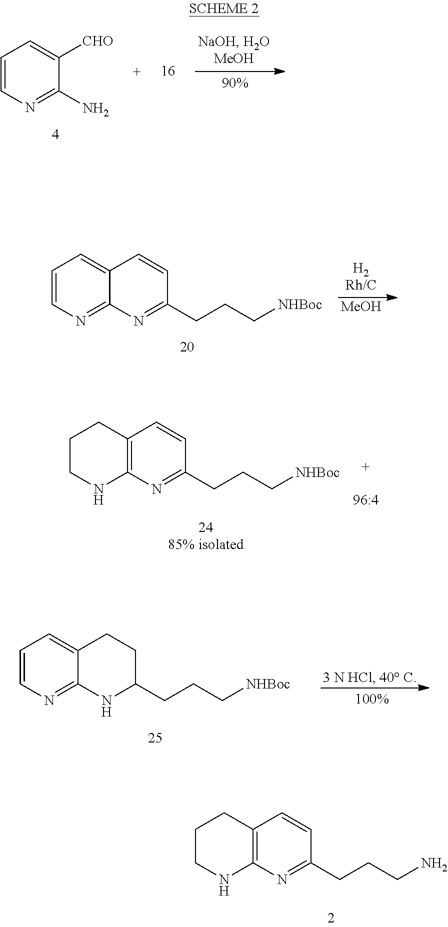

The modified Friedländer reaction of 4 and 16 proceeded smoothly in methanol with aqueous sodium hydroxide to provide naphthyridine 20 in 90% isolated yield. When the reaction was run in THF, compound 21 was produced. The next step was the partial reduction of 20 using Rh/C, 40 psi H₂, 5° C., MeOH, to provide a 96: 4 mixture of 24 and 25. After catalyst removal, compound 24 was crystallized from aqueous MeOH to provide material that was 99.8 wt % pure in 85% isolated yield. Deprotection of 24 proceeded smoothly in aqueous HCl and provided 2 in quantitative yield.

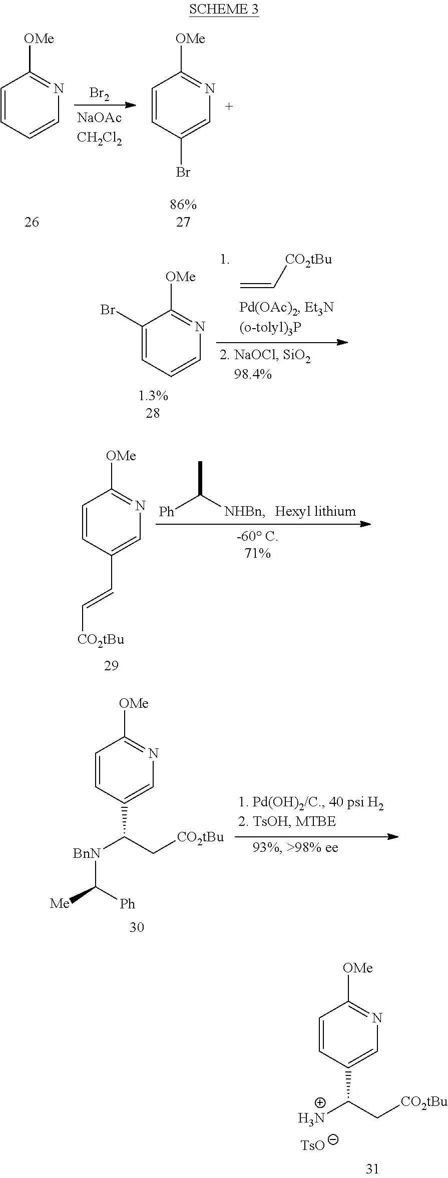

β-Alanine 3 was prepared as shown in Scheme 3, with Davies' chiral amine

Michael addition as the key reaction. (Davies, S. G.; Ichihara, O. Tetrahedron Asymmetry, 1991, 2, 183.)

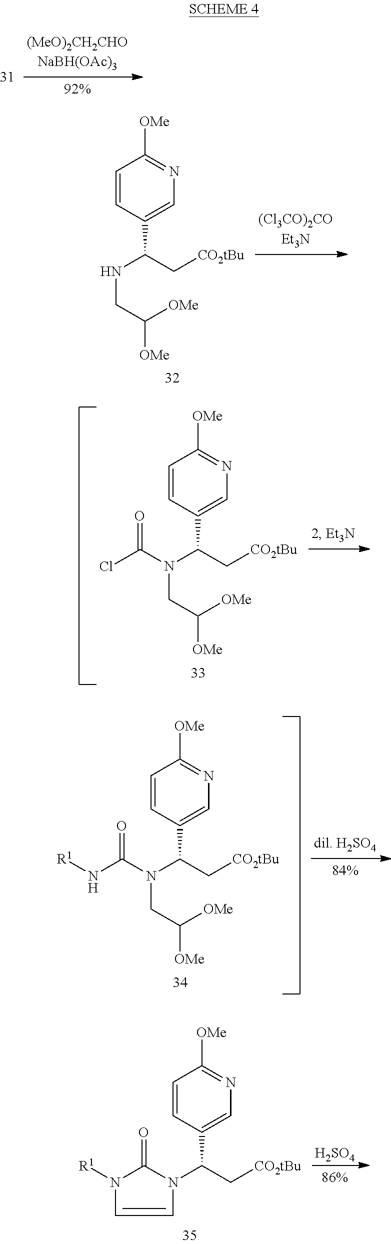

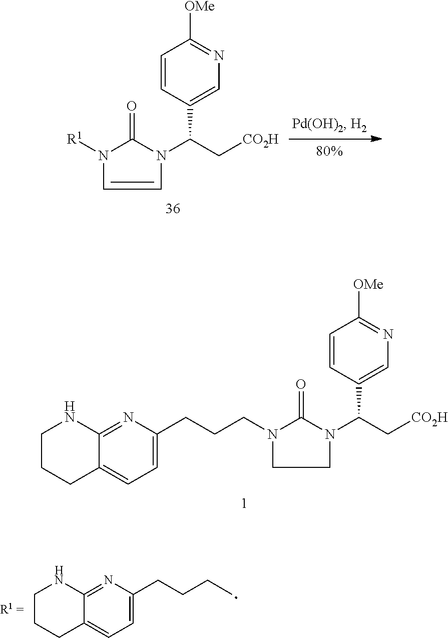

Reductive amination of 31 with dimethoxyacetaldehyde was followed by treatment with bis(trichloromethyl) carbonate.¹ Amine 2 is then introduced followed by cyclization. The optimized route is summarized in Scheme 4.

### EXAMPLE 1

### Preparation of Compound A

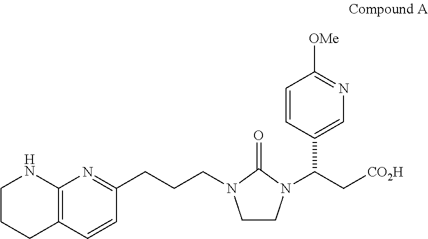

tert-Butyl 2-oxopyrrolidine-1-carboxylate (15). To a solution of 2-pyrrolidone (14, 33.8 mL, 444 mmol) and di-tert-butyl dicarbonate (97.0 g, 444 mmol) was added N,N-dimethylaminopyridine (92 mg, 0.75 mmol) and the mixture was stirred at 25-27° C. for 16 h. After the reaction was complete, the mixture was distilled at 40 mmHg, maintaining a constant volume by slow addition of toluene (100 mL). No tert-butanol was detected by GC or ¹H NMR. The resulting oil (86.0 g) contained 79.5 g of 15 (97% yield) and 7.6 wt % toluene. The solution was used for the next reaction without any further purification: ¹H NMR (400 MHz, CDCl₃) δ 3.72 (t, J=7.2 Hz, 2H), 2.48 (t, J=8.1 Hz, 2H), 1.97 (quintet, J=7.5 Hz, 2H), 1.50 (s, 9H); ¹³C NMR (100 MHz, CDCl₃) δ 174.2, 150.1, 82.6, 46.3, 32.8, 27.9, 17.3.

Dimethyl 5-[(tert-butoxycarbonyl)amino]-2-oxopentylphosphonate (16). To a solution of diisopropylamine (48.8 mL, 346 mmol) and dry THF (480 mL) was added hexyllithium (2.4M in hexanes, 133.6 mL, 320.6 mmol) below −10° C. After aged for 30 min, a solution of dimethyl methylphosphonate (65.2 mL, 333.4 mmol) in dry THF (128 mL) was slowly added to the reaction mixture, maintaining −60° C. After aged for 1 h at −60° C., a solution of 15 (50.0 g, 95 wt %, 256.5 mmol) and dry THF (32 mL) was slowly added, maintaining the reaction temperature below −58° C. The solution was stirred at −60° C. for 1 hour and at −45° C. for 1 h. To the solution was added sulfuric acid (2 N, 333.4 mL). The mixture was allowed to warm up to 0° C. The organic layer was separated and concentrated in vacuo. The residue was dissolved in methanol (150 mL) and used at the next reaction without further purification. The isolated yield was 80%. An analytical standard was prepared by silica gel column chromatography: ¹H NMR (400 MHz, CDCl₃) δ 5.05 (broad s, 1H), 3.62 (d, J=11.2 Hz, 6H), 2.96 (d, J=22.0 Hz, 2H), 3.00-2.90 (m, 2H), 2.51 (t, J=7.0 Hz, 2H), 1.60 (quintet, J=6.8 Hz, 2H), 1.26 (s, 9H); ¹³C NMR (101 MHz, CDCl₃) δ 200.9 (d, J=6.0 Hz), 155.5, 77.9, 52.3 (d, J=6.4 Hz), 40.6 (d, J=127.7 Hz), 40.3, 38.8, 27.7, 23.1.

tert-Butyl 3-(1,8-naphthyridin-2-yl)propylcarbamate (20). To a solution of 2-aminonicotinaldehyde (4, 21.8 g, 179 mmol) and β-keto phosphonate (16, 77.5 g, 95 wt %, 238 mmol) and methanol (400 mL) was added aqueous sodium hydroxide (50 wt %, 13.7 mL). The mixture was stirred at 40-50° C. for 30 min. Additional aldehyde 4 (5.4 g, 44 mmol) was added to the mixture with 100 mL of methanol. The mixture was stirred at 40-50° C. for 16 h. The mixture was concentrated in vacuo. The residue was partitioned between ethyl acetate (270 mL) and water (135 mL). The organic layer was washed with water (150 mL) and concentrated in vacuo. The residue was dissolved in methanol (300 mL) and used in next step without further purification. Assay of the methanol solution indicated a 90% yield. An analytical standard was prepared by silica gel column chromatography: ¹H NMR (400 MHz, CDCl₃) δ 8.98 (dd, J=4.2, 2.0 Hz, 1H), 8.07 (dd, J=8.1, 2.0 Hz, 1H), 8.01 (d, J=8.3 Hz, 1H), 7.35 (dd, J=8.1, 4.2 Hz, 1H), 7.31 (d, J=8.3 Hz, 1H), 4.93 (broad s, 1H), 3.15 (quartet, J=6.5 Hz, 2H), 3.00 (t, J=7.6 Hz, 2H), 2.03 (quintet, J=7.2 Hz, 2H), 1.34 (s, 9H); ¹³C NMR (101 MHz, CDCl₃) δ 165.7, 155.9, 155.7, 153.1, 137.0, 136.7, 122.5, 121.4, 120.9, 78.7, 39.9, 36.1, 29.1, 28.3.

tert-Butyl 3-(5,6,7,8-tetrahydro-1,8-naphthyridin-2-yl)propylcarbamate (24). A solution of naphthyridine 20 (2.72 g, 9.5 mmol) and methanol (20 mL) was hydrogenated in the presence of 5% rhodium on carbon (2.1 g, containing 63% of water) under 40 psi of hydrogen at 5° C. for 10 h. The catalyst was removed by filtration through Solka Floc and the filter cake was rinsed with methanol (2×25 mL). The filtrate and washings were combined, concentrated in vacuo, and dissolved in methanol (6.8 mL). To the combined filtrate was added water (6.8 mL) slowly at rt to induce crystallization. The resulting solid was collected by filtration, washed with a mixture of water and methanol (2:1, 5 mL), and dried under vacuum to give tetrahydronaphthyridine 24 (2.33 g, 85%). The mother liquor loss was 5%: mp 95.2-96.3° C.; ¹H NMR (400 MHz, CDCl₃) δ 7.05 (d, J=7.4 Hz, 1H), 6.33 (d, J=7.3 Hz, 1H), 5.45 (bs, 1H), 4.92 (bs, 1H), 3.39 (m, 2H), 3.16 (bm, 2H), 2.68 (t, J=6.2 Hz, 2H), 2.59 (t, J=7.3, 2H), 1.89 (m, 2H), 1.83 (m, 2H), 1.44 (s, 9H); ¹³C NMR (100.6 MHz, CDCl₃) δ 157.1, 156.0, 155.4, 136.7, 113.4, 111.3, 78.6, 41.4, 40.3, 35.0, 29.4, 28.4, 26.2, 21.3. Anal. Calcd for C₁₆H₂₅N₃O₂: C, 65.95; H, 8.65; N, 14.42. Found: C, 66.09; H, 8.62; N, 14.44.

5-Bromo-2-methoxypyridine (27). To a suspension of 2-methoxypyridine (26, 3.96 kg, 36.3 mol), NaOAc (3.57 kg, 39.9 mol), and dichloromethane (22 L) was added a solution of bromine (2.06 L, 39.9 mol) and dichloromethane (2 L), maintaining the reaction temperature below 7° C. over 2-3 h. The mixture was aged for 1 h at 0-7° C. and stirred at rt for 16 h. The reaction mixture was filtered and the filter cake was rinsed with dichloromethane (5 L). The filtrate and washings were combined, extracted with cold 2 MNaOH (22 L, pH should be below 8) maintaining the temperature below 10° C., and with cold water (11 L). The organic layer was concentrated under reduced pressure to give crude 27 (6.65 kg), which was purified by vacuum distillation to give pure 27 (5.90 kg, 86%) along with 1.3% of 28. 27: ¹H NMR (250 MHz, CDCl₃) δ 8.18 (d, J=2.5 Hz, 1H), 7.61 (dd, J=8.8, 2.5 Hz, 1H), 6.64 (d, J=8.8 Hz, 1H), and 3.89 (s, 3H); ¹³C NMR (62.9 MHz, CDCl₃) δ 162.9, 147.5, 141.0, 112.6, 111.7, 53.7. tert-Butyl(2E)-3-(6-methoxypyridin-3-yl)prop-2-enoate (29). A mixture of tert-butyl acrylate (137 mL, 916 mmol), triethylamine (100 mL, 720 mmol), tri-O-tolylphosphine (6.30 g, 20 mmol), Pd(OAc)₂ (1.80 g, 8 mmol), and NMP (90 mL) was degassed three times. The mixture was heated to 90° C. and a solution of 27 (50.0 g, 266 mmol) and NMP (10 mL) was added via addition funnel over 1 h, maintaining the reaction temperature at 90° C. After an additional 12 h at 90° C., the mixture was cooled to rt. Toluene (400 mL) was added and the resulting solution was passed through a pad of Solka Flok. The filter cake was washed with toluene (270 mL). The combined toluene solution was extracted with water (3×540 mL). An aqueous solution of NaClO (2.5%, 200 mL) was slowly added to the toluene solution keeping the temperature about 30° C. The reaction stirred vigorously for 50 min. The organic layer was separated, washed with water (3×540 mL), and saturated aqueous NaCl (270 mL). The organic layer was concentrated to oil. The oil was dissolved in hexanes (270 mL) and loaded onto to a silica gel pad (90 g). The silica gel pad was eluted with hexanes (73 mL) followed by EtOAc:hexane (1:8, v/v, 730 mL). The rich cut was concentrated to provide an oil (126 g, 49.2 wt %, 98.4% yield). The crude oil was used for the next reaction without further purification. An authentic crystalline sample was obtained by further concentration of the oil: mp 44-45° C.; ¹H NMR (250 MHz, CDCl₃) δ 8.23 (d, J=2.4 Hz, 1H), 7.73 (dd, J=8.7 and 2.4 Hz, 1H), 7.50 (d, J=16.0 Hz, 1H), 6.73 (d, J=8.7 Hz, 1H), 6.25 (d, J=16.0 Hz, 1H), 3.94 (s, 3H), and 1.51 (s, 9H); ¹³C NMR (62.9 MHz, CDCl₃) δ 166.1, 165.1, 148.1, 139.9, 136.3, 124.0, 119.1, 111.5, 80.6, 53.7, and 28.2. Anal. Calcd for C₁₃H₁₇NO₃: C, 66.36; H, 7.28; N, 5.95. Found: C, 66.35; H, 7.43; N, 5.79.

tert-Butyl(3S)-3-{benzyl[(1R)-1-phenylethyl]amino}-3-(6-methoxypyridin-3-yl)propanoate (30). To a solution of (R)-(+)-N-benzyl-α-methylbenzylamine (88 mL, 0.42 mol) and anhydrous THF (1 L) was added n-BuLi (2.5 M in hexanes, 162 mL, 0.41 mol) over 1 h at −30° C. The solution was cooled to −65° C. and a solution of t-butyl ester 29 (65.9 g, 0.28 mol) and anhydrous THF (0.5 L) was added over 90 min during which the temperature rose to −57° C. After the reaction was complete, the reaction solution was poured into a mixture of saturated aqueous NH₄Cl (110 mL) and EtOAc (110 mL). The organic phase was separated, washed sequentially with aqueous AcOH (10%, 110 mL), water (110 mL) and saturated aqueous NaCl (55 mL). The organic layer was concentrated in vacuo to provide a crude oil. The crude oil was purified by passing through a silica gel (280 g) pad eluting with 95:5 hex/EtOAc. The product containing fractions were combined and concentrated in vacuo to give an oil. The resulting oil was used directly in the next step. The oil contained 91 g (0.20 mol, 71%) of the product 30: ¹H NMR (400 MHz, CDCl₃) δ 8.16 (d, J=2.4 Hz, 1H), 7.65 (dd, J=8.8, 2.4 Hz, 1H), 7.40 (m, 2H), 7.34 (m, 2H), 7.30-7.16 (m, 6H), 6.74 (d, J=8.8 Hz, 1H), 4.39 (dd, J=9.8, 5.3 Hz, 1H), 3.97 (q, J=6.6 Hz, 1H), 3.94 (s, 3H), 3.67 (s, 2H), 2.52 (dd, J=14.9, 5.3 Hz, 1H), 2.46 (dd, J=14.9, 9.8 Hz, 1H), 1.30 (d, J=6.6 Hz, 3H), 1.26 (s, 9H); ¹³C NMR (101 MHz, CDCl₃) δ 170.8, 163.3, 146.4, 143.8, 141.3, 138.6, 130.0, 128.24, 128.19, 127.9, 127.7, 127.0, 126.6, 110.4, 80.5, 57.4, 56.6, 53.4, 50.7, 37.5, 27.8, 17.3. Anal. Calcd for C₂₈H₃₄N₂O₃: C, 75.31; H, 7.67; N, 6.27. Found: C, 75.13; H, 7.75; N, 6.17.

tert-Butyl(3S)-3-amino-3-(6-methoxypyridin-3-yl)propanoate 4-methylbenzenesulfonate (31). The thick oil (30, containing 80.3 g, 0.18 mol) was hydrogenated in the presence of Pd(OH)₂ (20 wt % on carbon, 8.0 g) in a mixture of EtOH (400 mL), AcOH (40 mL) and water (2 mL) under 40 psi of hydrogen at 35° C. for 8 h. The reaction mixture was filtered through a pad of Solka Flok, evaporated to a thick oil in vacuo. MTBE (2 L) was added and the resulting solution was evaporated to provide an oil. This was repeated several times. A hot solution (40° C.) ofp-toluenesulfonic acid (p-TsOH, 41.7 g, 0.22 mol) and MTBE (400 mL) was added slowly to the warm solution of the amine. After ˜30% of the p-TsOH solution had been added, the solution was seeded and a thick slurry formed. The remaining p-TsOH was added over 2 h. The resulting suspension was aged for 3 h at 45° C. The suspension was then slowly cooled to room temperature. After 12 h at room temperature the mixture was cooled to 6° C. The solids were collected on a frit, rinsed with MTBE (100 mL) and dried under vacuum at 35° C. to give 31 (71.0 g, 93%, >98% ee): mp 142-144° C.; ¹H NMR (400 MHz, CDCl₃) δ 8.40 (bs, 3H), 8.22 (s, 1H), 7.87 (d, J=8.8 Hz, 1H), 7.56 (d, J=8.0 Hz, 2H), 7.11 (d, J=8.0 Hz, 2H), 6.65 (d, J=8.8 Hz, 1H), 4.63 (m, 1H), 3.91 (s, 3H), 3.09 (dd, J=16.5 and 6.0 Hz, 1H), 2.87 (dd, J=16.5, 8.8 Hz, 1H), 2.36 (s, 3H), 1.27 (s, 9H); ¹³C NMR (101 MHz, CDCl₃) δ 168.4, 164.2, 146.8, 140.9, 140.4, 137.8, 128.8, 125.8, 124.3, 111.0, 81.6, 53.5, 49.6, 39.3, 27.8, 21.3. Anal. Calcd for C₂₀H₂₈N₂O₆S: C, 56.59; H, 6.65; N, 6.60; S, 7.55. Found: C, 56.61; H, 6.76; N, 6.56; S, 7.59.

tert-Butyl(3S)-3-[(2,2-dimethoxyethyl)amino]-3-(6-methoxypyridin-3-yl)propanoate (32). To a solution of 31 (100 g, 239 mmol) and dimethoxyacetaldehyde (60 wt % in water, 39.3 mL, 261 mmol) and THF (400 mL) was added a suspension of sodium triacetoxyborohydride (95 wt %, 79 g, 354 mol) and THF (200 mL) over 1 h, maintaining the reaction temperature below 10° C. The residual sodium triacetoxyborohydride was rinsed into the reaction mixture with THF (40 mL). The mixture was stirred at 5-10° C. for 30 min and then at room temperature for 30 min. After cooling to below 10° C., aq. Na₂CO₃ (10 wt %, 120 mL) was added maintaining the temperature below 10° C. The mixture was extracted with EtOAc (750 mL) and the organic phase was washed with sat. aq. NaHCO₃ (600 mL) and water (500 mL), and concentrated in vacuo to give crude 32 (88.4 g, 83.9 wt %, 92.2%). An analytical sample was prepared by silica gel column chromatography: ¹H NMR (400 MHz, CDCl₃) δ 8.08 (d, J=2.4 Hz, 1H), 7.61 (dd, J=8.4, 2.4 Hz, 1H), 6.73 (d, J=8.4 Hz, 1H), 4.41 (t, J=5.6 Hz, 1H), 4.00 (dd, J=8.2, 6.0 Hz, 1H), 3.93 (s, 3H), 3.35 (s, 3H), 3.31 (s, 3H), 2.67 (dd, J=15.3, 8.2 Hz, 1H), 2.60 (dd, J=12.0, 5.6 Hz, 1H), 2.51 (dd, J=12.0, 5.6 Hz, 1H), 2.49 (dd, J=15.3, 6.0 Hz, 1H), 1.40 (s 9H); ¹³C NMR (101 MHz, CDCl₃) δ 170.6, 163.8, 145.9, 137.4, 130.4, 110.9, 103.5, 80.9, 56.9, 53.71, 53.68, 53.4, 48.6, 43.8, 28.0.

tert-Butyl(3S)-3-(6-methoxypyridin-3-yl)-3-{2-oxo-3-[3-(5,6,7,8-tetrahydro-1,8-naphthyridin-2-yl)propyl]-2,3-dihydro-1H-imidazol-1-yl}propanoate (35). A solution of 24 (10.4 g, 35 mmol) and 6 M HCl (18 mL) was stirred at 35° C. for 1.5 h. The pH of the reaction mixture was adjusted at 7 with 50 wt % NaOH. After addition of sec-butanol (35 mL), the pH of the aqueous layer was adjusted at 11.5 with 50 wt % NaOH. The organic layer was separated, washed with sat. aq. NaCl (10 mL), and concentrated in vacuo to remove water to yield a dry solution of amine 2 (35 mmol) and sec-butanol.

A solution of 32 (10 g as pure, 29 mmol), triethylamine (5.5 mL, 40 mmol) and THF (45 mL) was added to a cold solution of bis(trichloromethyl)carbonate (3.51 g, 12 mmol) and THF (75 mL) over 30 min, maintaining the temperature below 0° C. The mixture was stirred for 2 h at room temperature to yield chlorocarbamate 33. The solution of 2, prepared above, and triethylamine (5.5 mL, 40 mmol) was added to the reaction mixture containing 33. The resulting mixture was stirred at 45° C. for 3 h. To the mixture was added water (20 mL). The phases were separated and the organic layer, which contained urea 34, was retained. To the organic layer was added 2 M sulfuric acid (40 mL) and the mixture was stirred for 18 h at room temperature. To the mixture was added iPAc (50 mL). The organic layer was separated and extracted with 2M sulfuric acid (20 mL). The aqueous layers were combined and extracted with iPAc (50 mL). iPAc (80 mL) was added to the aqueous phase and the two phase mixture was cooled to 0° C. The pH was adjusted to 8.3 by addition of 5 M NaOH (˜40 mL). The organic layer was separated and washed with water (3×45 mL). The solution containing 35 (12.0 g, 84%) was used for the next step without further purification. An analytical sample was prepared by silica gel column chromatography: ¹H NMR (250 MHz, CDCl₃) δ 8.05 (d, J=2.5 Hz, 1H), 7.53 (dd, J=8.6, 2.5 Hz, 1H), 6.95 (d, J=7.3 Hz, 1H), 6.63 (d, J=8.6 Hz, 1H), 6.25 (d, J=7.3 Hz, 1H), 6.16 (d, J=3.0 Hz, 1H), 6.12 (d, J=3.0 Hz, 1H), 5.53 (t, J=8.1 Hz, 1H), 4.90 (bs, 1H), 3.82 (s, 3H), 3.54 (t, J=7.1 Hz, 2H), 3.32-3.23 (m, 2H), 3.04 (dd, J=15.5, 8.3 Hz, 1H), 2.90 (dd, J=15.5, 7.9 Hz, 1H), 2.59 (t, J=6.3 Hz, 2H), 2.46 (t, J=7.5 Hz, 2H), 1.93 (m, 2H), 1.80 (m, 2H), 1.27 (s, 9H); ¹³C NMR (62.9 MHz, CDCl₃) δ 168.6, 163.6, 156.6, 155.5, 152.1, 145.1, 137.6, 136.5, 127.6, 113.2, 111.1, 110.8, 110.7, 107.4, 81.1, 53.3, 51.2, 42.8, 41.3, 39.6, 34.4, 29.1, 27.6, 26.1, 21.2. Anal. Calcd for C₂₇H₃₅N₅O₄: C, 65.70; H, 7.15; N, 14.19.

(3S)-3-(6-Methoxypyridin-3-yl)-3-{2-oxo-3-[3-(5,6,7,8-tetrahydro-1,8-naphthyridin-2-yl)propyl]-2,3-dihydro-1H-imidazol-1-yl}propanoic acid (36). To a solution of 35 and iPAc (140 mg/mL, 220 mL, 30.8 g, 62.4 mmol) was added 3.06 M sulfuric acid (150 mL). The aqueous layer was separated and stirred at 40° C. for 3 h. The mixture was cooled to 10° C. The pH of the solution was adjusted to about 2 with 50 wt % NaOH. To the solution was added SP207 resin¹ (310 mL). The pH of the suspension was adjusted to 5.9 with 50 wt % NaOH and stirred at room temperature for 4 h. The suspension was filtered and the resin was washed with water (930 mL) and then with 70 v/v % acetone-water (1.5 L). The fractions containing 36 were combined and concentrated to remove acetone. The resulting suspension was cooled to 5° C. Crystals were collected by filtration, washed with cold water (20 mL), and dried at 30° C. under vacuum to provide 36 (23.5 g, 86%) as crystals. Recrystallization from aq. iPA gave a thermodynamically more stable crystal form: mp 123° C.; ¹H NMR (400 MHz, CD₃OD) δ 8.16 (d, J=2.6 Hz, 1H), 7.73 (dd, J=8.6, 2.6 Hz, 1H), 7.45 (d, J=7.4 Hz, 1H), 6.81 (d, J=8.6 Hz, 1H), 6.54 (d, J=3.1 Hz, 1H), 6.53 (d, J=7.4 Hz, 1H), 6.50 (d, J=3.1 Hz, 1H), 5.70 (dd, J=11.6, 4.2 Hz, 1H), 3.90 (s, 3H), 3.76 (ddd, J=14.0, 9.7, 4.3 Hz, 1H), 3.51 (dt, J=14.0, 5.0 Hz, 1H), 3.46 (m, 2H), 2.99 (dd, J=14.0, 11.6 Hz, 1H), 2.85 (dd, J=14.0, 4.2 Hz, 1H), 2.77 (t, J=6.2 Hz, 2H), 2.70 (ddd, J=13.5, 7.5, 5.3 Hz, 1H), 2.50 (dt, J=15.3, 8.2 Hz, 1H), 2.14-1.87 (m, 4H); ¹³C NMR (101 MHz, CD₃OD) δ 177.6, 163.9, 153.8, 152.2, 148.8, 145.0, 140.1, 137.9, 128.6, 118.2, 111.1, 110.4, 109.5, 108.6, 52.7, 52.1, 41.5, 40.8, 40.3, 28.9, 28.1, 25.1, 19.4. Anal. Calcd for C₂₃H₂₇N₅O₄.0.5 H₂O: C, 61.87; H, 6.30; N, 15.64. Found C, 61.76; H, 6.12; N, 15.71. KF 1.97%.

(3S)-3-(6-Methoxypyridin-3-yl)-3-{2-oxo-[3-[3-(5,6,7,8-tetrahydro-1,8-naphthyridin-2-yl)propyl]imidazolidin-1-yl}propionic acid (1). A suspension of 36 (105 g, 240 mmol), water (247 mL), 5 M NaOH (84 mL) and 20 wt % Pd(OH)₂/C (21 g) was hydrogenated at 120 psi of hydrogen at 80° C. for 18 h. The pH was adjusted to 9.0 with conc. HCl and the catalyst was removed by filtration through a pad of Solka Flok (13 g). The filter cake was rinsed with water (200 mL) and the combined filtrate was adjusted to pH 6.4 with conc. HCl. The solution was seeded and stirred at 0° C. for 1 h. The resulting crystals were collected by filtration and dried under nitrogen to provided 1 as a hemihydrate (84.5 g, 80%): mp 122° C.; ¹H NMR (500 MHz, CD₃OD) δ 8.08 (d, J=2.4 Hz, 1H), 7.66 (dd, J=8.7, 2.4 Hz, 1H), 7.45 (d, J=7.2 Hz, 1H), 6.79 (d, J=8.7 Hz, 1H), 6.53 (d, J=7.2 Hz, 1H), 5.48 (dd, J=12.3, 3.6 Hz, 1H), 3.89 (s, 3H), 3.64 (q, J=9.2 Hz, 2H), 3.50 (m, 1H), 3.45 (m, 2H), 3.34 (ddd, J=14.1, 12.1, 3.9 Hz, 1H), 3.16 (q, J=9.1 Hz, 1H), 2.98 (m, 1H), 2.97 (t, J=12.3 Hz, 1H), 2.81 (dt, J=14.1, 4.0 Hz, 1H), 2.75 (m, 3H), 2.65 (ddd, J=14.4, 11.2, 5.0 Hz, 1H), 2.55 (dd, J=12.3, 3.4 Hz, 1H), 2.06 (m, 1H), 1.92 (m, 2H), 1.82 (m, 1H); ¹³C NMR (125.7 MHz, CD₃OD) δ 180.7, 165.1, 162.6, 153.3, 150.2, 146.6, 141.4, 139.7, 130.0, 119.6, 111.6, 110.7, 54.1, 53.1, 42.2, 41.6, 41.0, 38.7, 38.6, 29.1, 27.9, 26.6, 20.7. Anal. Calcd for C₂₃H₂₉N₅O₄: C, 62.85; H, 6.65; N, 15.94. Found C, 62.51; H, 6.76; N, 16.04

### EXAMPLE 2

### Dimethyl-Phosphinic Acid C-43 Rapamycin Ester

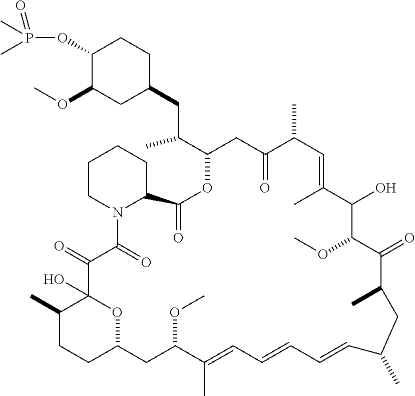

To a cooled (0° C.) solution of rapamycin (0.1 g, 0.109 mmol) in 1.8 mL of dichloromethane was added 0.168 g (0.82 mmol) of 2,6-di-t-butyl-4-methyl pyridine, under a stream of N₂, followed immediately by a solution of dimethylphosphinic chloride (0.062 g, 0.547 mmol) in 0.2 mL of dichloromethane. The slightly yellow reaction solution was stirred at 0° C., under an atmosphere of N₂, for 3.5 h (reaction monitored by TLC). The cold (0° C.) reaction solution was diluted with ˜20 mL EtOAc then transferred to a reparatory funnel containing EtOAc (150 mL) and saturated NaHCO₃ (100 mL). Upon removing the aqueous layer, the organic layer was washed successively with ice cold 1N HCl (1×100 mL), saturated NaHCO₃(1×100 mL), and brine (1×100 mL), then dried over MgSO₄ and concentrated. The crude product was purified by silica gel flash chromatography (eluted with 1:10:3:3 MeOH/DCM/EtOAc/hexane) to provide 0.092 g of a white solid: ¹H NMR (300 MHz, CDCl3) δ4.18 (m, 1H), 4.10 (m, 1H), 3.05 (m, 1H), 1.51 (m, 6H); ³¹P NMR (121 MHz, CDCl₃) δ 53.6; 1013 m/z (M+Na).

### EXAMPLE 3

### Dimethyl-Phosphinic Acid C-43 Rapamycin Ester, Alternative Synthesis

Rapamycin and dichloromethane are charged into a nitrogen-purged reaction flask. The stirred solution is cooled to approximately 0° C. (an external temperature of −5±5° C. is maintained throughout the reaction). A solution of dimethylphosphinic chloride (2.0 molar equivalents) in dichloromethane is then added over a period of approximately 8-13 minutes. This is followed immediately by the addition of a solution of 3,5-lutidine (2.2 molar equivalents) in dichloromethane over a period of approximately 15-20 minutes. Throughout both additions, the internal temperature of the reaction stays below 0° C. The cooled reaction solution is stirred for 1 hour and then transferred, while still cold, to an extractor containing saturated aqueous NaHCO₃ and methyl-t-butyl ether (MTBE), ethyl acetate or diethyl ether. In-process samples are removed at 30 and 60 minute time points. Samples are prepared in a similar fashion to that described for the reaction workup. Reaction progress is monitored by TLC (1:10:3:3 MeOH/DCM/EtOAc/hexanes) and reverse-phase HPLC analyses. The isolated organic layer is successively washed with ice cold 1N HCl, saturated aqueous NaHCO₃ (2×), saturated aqueous NaCl, and dried over sodium sulfate. Upon filtration and solvent removal, the residue undergoes solvent exchange with acetone followed by concentration in vacuo to provide crude product, which may be analyzed for purity by normal- and reversed-phase HPLC.

### EXAMPLE 4

### Effect of Compound a and Ridaforolimus in Human Cancer Cell Lines

Summary: Rationale for the proposed combination is based on the results from a whole genome siRNA screen in which ITGAV knockdown inhibited the ridaforolimus induced activation of Akt.

Ridaforolimus is currently being developed for the treatment of lung cancer. Treatment with rapamycin analogues results in the up-regulation of AKT signaling as measured by phosphorylation of AKT. While inhibition of mTOR by Ridaforolimus can induce tumor growth arrest, it abrogates a negative feedback loop mediated by IRS-1, resulting in activation of AKT, which has been implicated in reducing its anti-tumor activity. A recent clinical study suggests that activation of AKT via this feedback mechanism may be associated with a shorter time-to-progression in patients treated with rapamycin (Cloughesy et al PLoS Medicine, 2008). We have found that knockdown of ITGAV inhibits the negative feedback loop induced by ridaforolimus thus by combining ridaforolimus with an integrin alpha V inhibitor may be beneficial for inhibiting the PI3K pathway as well as enhancing anti-tumor activity of ridaforolimus. To investigate this possibility, the inventors examined the proposed combination in a panel of cancer cell lines. Detailed here below is data supporting the hypothesis that the combination treatment comprising Ridaforolimus and Compound A significantly enhanced inhibition of cell proliferation.

### (A) Compound A+Ridaforolimus Combination Enhances Inhibition of Cell Proliferation:

| | Rida/ | |
|---|---|---|
| Cell Line | Cmpd A VHSA | indication |
| HT1080 | 0.08 | sarcoma |
| MCF7 | 0.11 | breast |
| MDA-MB- | 0.14 | breast |
| 415 | | |
| ZR-75-1 | 0.15 | breast |
| A549 | 0.1 | lung |
| EBC-1 | 0.03 | lung |
| H520 | 0.16 | lung |
| H292 | 0.019 | lung |
| H1703 | 0.16 | lung |
| H2122 | 0.037 | lung |
| H322 | −0.02 | lung |

VHSA
<0 antagonistic
=0 additive
>0 synergistic
≧0.1 true synergy
≧0.2 strongly synergistic

Methods: Proliferation assays were conducted in 96 well plates with cells were seeded at a concentration of 3500 cells per well. The highest concentration of ridaforolimus was 50 nM and the highest concentration of Compound A was 30 μM. Each compound was diluted 1:3 for eight points. Twenty-four hours after seeding the cells, an eight by eight matrix of the two compound dose curves was added to the cells. Cells were incubated for 72 hours and then a Vialight assay (Lonza) was performed to determine cell number.
Analysis: The Highest Single Agent (HSA) method was used to determine if the combination was synergistic in each of the cell lines tested.

While a number of embodiments of this invention have been described, it is apparent that the basic examples may be altered to provide other embodiments, encompassed by the present invention. Therefore, it will be appreciated that the scope of this invention is to be defined by the appended claims rather than by the specific embodiments, which have been represented by way of example.

What is claimed is:

1. A method of treating a cancer selected from the group consisting of non-small cell lung cancer and breast cancer with an mTOR inhibitor and an αvβ3 integrin antagonist, wherein the mTOR inhibitor is selected from the group consisting of ridaforolimus, everolimus, temsirolimus and combinations thereof, and the αvβ3 integrin antagonist is a compound of structural formula I: 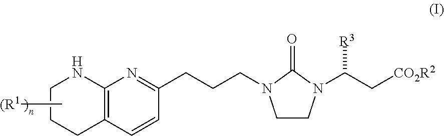
wherein each R¹ is independently selelcted from the group consisting of hydrogen, C₁₋₄ alkyl and cyclopropyl; or two R¹ substituents, when on the same carbon atom, are taken together with the carbon atom to which they are attached to form a spirocyclopropyl group;
R² is hydrogen or C₁₋₄ alkyl;
R³ is mono-or di-substituted quinolinyl, pyridinyl or pyrimidinyl; wherein the substituents are each independently selected from the group consisting of hydrogen, halo, phenyl, C₁₋₄ alkyl, C₃₋₆ cycloalkyl, C₁₋₃ alkoxy, amino, C₁₋₃ alkylamino, di(C₁₋₃ alkylamino), hydroxyl, cyano, trifluoromethyl, trifluoroethyl, trifluoromethoxy and trifluoroethoxy.

2. The method of claim 1 wherein the mTOR inhibitor is ridaforolimus.

3. The method of claim 2 wherein the αvβ3 integrin antagonist is

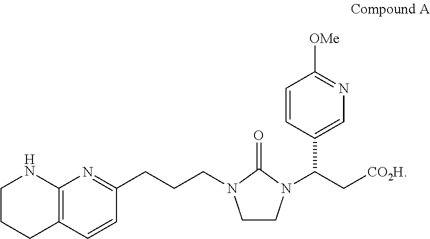

4. The method of claim 1 wherein the mTOR inhibitor is ridaforolimus and the αvβ3 integrin antagonist is

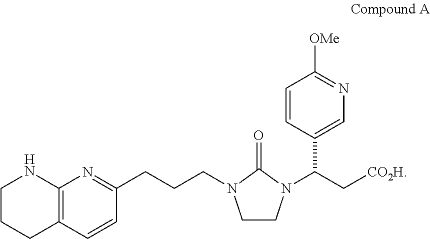

5. The method of claim 4 wherein ridaforolimus is administered in a dose between 10 mg and 40 mg.

6. The method of claim 5 wherein ridaforolimus is administered five times a week.

7. The method of claim 4 wherein Compound A is administered in a dose between 200 mg and 1600 mg per day.
